(19) United States
(12) Patent Application Publication
(10) Pub. No.: US 2026/0286135 A1
(43) Pub. Date: Sep. 24, 2026

(54) OPHTHALMIC LENS COMPRISING AN ACRYLONITRILE COMPOUND

(71) Applicant: ESSILOR INTERNATIONAL, CHARENTON-LE-PONT (FR)

(72) Inventors: Supitchaya IAMSAARD, CHARENTON LE PONT (FR); Prakhar KASTURE, CHARENTON LE PONT (FR); Pierre FROMENTIN, CHARENTON LE PONT (FR)

(21) Appl. No.: 19/473,899

(22) PCT Filed: Apr. 26, 2024
(86) PCT No.: PCT/EP2024/061637

§ 371 Date: Oct. 9, 2025

(30) Foreign Application Priority Data
Apr. 28, 2023 (EP) 23315139.8

(51) Int. Cl. C09B 57/00; C07D 207/337; C08G 18/38; C08G 18/75; C08G 18/76; C08K 5/3415; C08K 5/3475; C09B 67/20; G02B 1/04

(52) U.S. Cl. CPC C09B 57/00; C07D 207/337; C08G 18/3876; C08G 18/758; C08G 18/7642; C08K 5/3415; C08K 5/3475; C09B 67/0063; G02B 1/041

(57) ABSTRACT

The present invention relates to ophthalmic lenses comprising a compound of Formula (I), a preparation method thereof and the use of a compound of Formula (I) in an ophthalmic lens to absorb blue light.

### TECHNICAL FIELD

This disclosure relates to ophthalmic lenses comprising an acrylonitrile compound, a method for producing said lenses, and the use of said compound in an ophthalmic lens to absorb blue light.

### BACKGROUND

Light that reaches and enters the human eye is divided into visible light, comprising wavelengths from about 380 to 780 nm, and non-visible light, which includes light in the ultraviolet range (UV-A and UV-B light from about 280 to 380 nm) and the infrared range (Near IR light from about 780 to 1400 nm).

UV light is known to be harmful to the human eye. In particular, it can accelerate ocular ageing which can lead to an early cataract or to more extreme disorders such as photokeratitis or «snow blindness».

Blue light, also known as high-energy visible (HEV) light, corresponds to visible light in the blue-violet band between 380 and 500 nm. Prolonged exposure to blue light emitted from digital devices such as television, laptops, tablets and smartphones and fluorescent and LED lighting is harmful as blue light is able to reach the retina. Some specific ranges of blue light have been shown to cause photoretinitis; digital eyestrain, or computer vision syndrome which includes blurry vision, difficulty focusing, dry and irritated eyes, headaches, neck and back pain; disruption of the circadian decreased melanin production; age-related macular degeneration; glaucoma; retinal degenerative diseases; breast and prostate cancer, diabetes; heart disease, obesity and depression.

Blue light in the range from about 420 to 450 nm is believed to be especially harmful.

Damages from UV light and blue light can be prevented by incorporating light-absorbing additives in ophthalmic lenses.

Three different methods can be used to prepare light-absorbing ophthalmic lenses. The first method is the impregnation of a polymerized lens in a bath containing a light-absorbing additive. However, this method adds a step to the production process of the lens, which is not desirable in terms of cost and time.

The second method is the coating of a substance capable of absorbing light rays onto the surface of ophthalmic lenses. However, the incorporation of high amounts of light-absorbing additives in a coating weakens its mechanical properties.

The third method is the incorporation of a light-absorbing additive in the bulk liquid formulation (i.e. before polymerization). However, the incorporation of high amounts of light-absorbing additives in the bulk can lead to undesirable yellowing as well as compatibility issues and degradation of mechanical properties.

Nowadays, the best commercial compounds for use in lenses are benzotriazoles and benzophenones. The main problem associated with commercial compounds stems from the fact that their spectral properties are not well adapted to blue cut use: typically, their maximum absorption wavelength (λₘₐₓ) is lower than that would be necessary to achieve significant extinction within blue-cut range. Indeed, most of the products available on the market exhibit a maximum absorption wavelength (λₘₐₓ) below or equal to 350 nm, leading to very poor or no absorption at, and above, 400 nm. Other compounds exhibit a maximum absorption wavelength within blue-cut range but suffer from low absorption extinction coefficients, i.e. lower than 30,000 M⁻¹cm⁻¹, at these wavelengths.

Such mismatch between spectral properties and application in blue cut leads to two consequences:
when blue cutting molecules are introduced in a volume having a thin dimension such as film or coating, the concentration required to reach blue cut makes it unpractical or not feasible;
when blue cutting molecules are introduced in the bulk, the concentration required to reach blue cut may cause compatibility issues such as blooming (the additive migrates out of the lens during polymerization and causes surface defects) or degradation of the thermo-mechanical properties of the matrix.

There is thus a need for blue cutting molecules that are efficient in low amounts to limit degradation of the mechanical properties of the polymer matrix and compatibility issues.

In particular, a need exists for robust lenses whose optical properties remain stable over time.

The Applicant has found that this need could be met with an ophthalmic lens comprising a compound of Formula (I) as defined herein as a light absorbing additive. Indeed, the compound of Formula (I) may have a maximum wavelength absorption in the range of from 350 to 400 nm and may have high absorption extinction coefficients, i.e. equal to or higher than 30,000 M⁻¹cm⁻¹, at these wavelengths and can therefore be used to absorb blue light more efficiently than existing commercial lenses comprising for instance benzotriazoles and benzophenones.

Ophthalmic lenses comprising a compound of Formula (I) show a higher blue cut performance, represented by BVC B′ or UV_{cut}, than commercial lenses comprising the same amount of a conventional benzotriazole; and furthermore, show low undesired yellowing and good clarity.

Since compounds of Formula (I) are more efficient for blue light cutting applications, the amount of light absorber that needs to be introduced in the lens is reduced, so that migration of light absorber at the surface of the lens during polymerization is minimized, resulting in lower contamination of mold surfaces and eventually eliminating the need to clean molds between successive uses. The use of reduced amounts of blue cutting molecules might decrease the cost of lens production.

In addition, substrates comprising low amounts of compound of Formula (I) will exhibit enhanced thermo-mechanical properties, because the mechanical properties of a substrate are usually improved when additives and/or adjuvants are used in lower amount.

The lenses according to the invention show moreover high light resistance and maintain optical properties over time.

Further, compounds of Formula (I) can also be used in thin dimension applications such as light-filtering coatings, hardcoats, films and laminates.

Additionally, compound of Formula (I) can be encapsulated in nanoparticles and lead to reduced yellowing in allylic matrixes requiring high amounts of catalyst to initiate polymerization.

Last, there is also a need for molecules that are efficient to filter a limited range of visible light, so as to protect wearer from harmful light or to improve vision of wearer, in particular to improve color perception, contrast or color discrimination (for colorblind wearers).

The Inventors found that the combination of optical properties of the ophthalmic lenses according to the invention, make them particularly useful in optical applications.

### SUMMARY

A first object of the present invention is an ophthalmic lens comprising:
a plastic base, and
a compound of Formula (I), or a salt thereof,

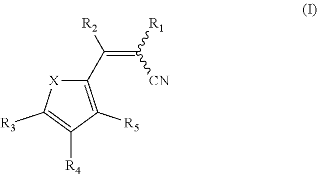

X, R₁, R₂, R₃, R₄ and R₅ being as defined herein.

A second object of the present invention is a process for preparing the ophthalmic lens of the invention, comprising the steps of:
a) providing monomers or oligomers from which the plastic base can be prepared,
b) mixing the monomers or oligomers, the compound of Formula (I) as defined herein, and a catalyst suitable for the polymerization of the monomers and oligomers to form a polymerizable liquid composition,
c) curing the polymerizable liquid composition.

A third object of the present invention is the use of a compound of Formula (I) as defined herein in an ophthalmic lens to absorb blue light.

A fourth object of the present invention is a composition comprising a plastic base and a compound of Formula (IA)

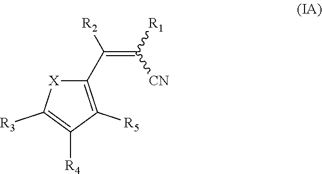

R₁, R₂, R₃, R₄ and R₅ being as defined herein.

A fifth object of the present invention is the use of a composition as defined herein in an optical article.

A sixth object of the present invention is the provision of novel compounds chosen from:

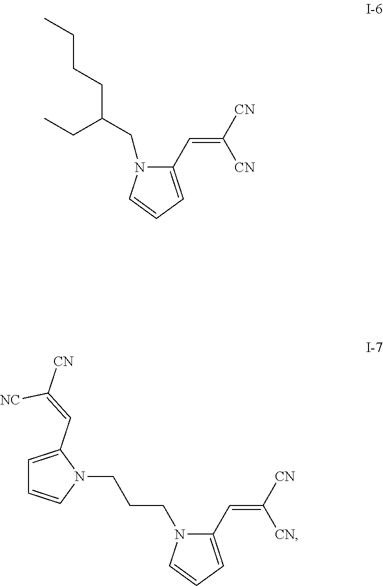 [SMILES: CCCCC(CC)Cn1cccc1C=C(C#N)C#N ; N#CC(C#N)=Cc1cccn1CCCn1cccc1C=C(C#N)C#N]

or salts thereof.

### DETAILED DESCRIPTION

### Ophthalmic Lens

The present invention relates to an ophthalmic lens.

As used herein, the term “ophthalmic lens” refers to any type of lens intended to be supported by a wearer's face, which may be for purposes of improving or enhancing visual acuity, for protecting against the environment, for fashion, or for adornment. The term may refer to ophthalmic lenses, such as non-corrective lenses (also called plano lenses), semi-finished lens blanks, and corrective lenses, such as progressive addition lenses, unifocal or multifocal lenses. Further examples of ophthalmic lens include electronic lens, virtual reality (VR) lens, augmented reality lenses (AR) and the like.

The ophthalmic lens of the present invention comprises a compound of Formula (I) or a salt thereof,

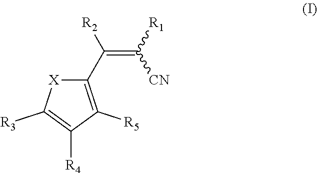

R₁ is chosen from —CN, —CONH₂, —CO₂R₆ or —SO₂—(C₁-C₁₂)alkyl,
R₂ is, independently from R₃, R₄ and R₅, chosen from H, F, Cl, Br, I, —OH, —OR₇, —SH, —SR₈, —SOR₉, —SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₃, R₄ and R₅ are independently chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
X is NH, NR₁₅, O, or S,
R₆, R₇, R₈, R₉, R₁₀, R₁₁, R₁₂, R₁₃ and R₁₄ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl, R₁₅ is chosen from (C₁-C₁₂)alkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl, (5 to 10 membered)heteroaryl-(C₁-C₁₂)alkyl, said alkyl being optionally substituted by one to three R₁₆ groups, R₁₆ is chosen from halogen atoms, (C₁-C₁₂)alkoxy, (C₆-C₁₈)aryl, or wherein R₁₅ is a structure of Formula a:

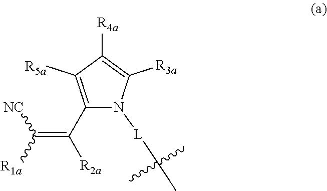

In which:
L is (C₁-C₁₂)alkylene,
R₁ₐ is chosen from —CN, —CONH₂, —CO₂R₆ₐ or —SO₂—(C₁-C₁₂)alkyl, R₂ₐ is, independently from R₃ₐ, R₄ₐ and R₅ₐ, chosen from H, F, Cl, Br, I, —OH, —OR₇ₐ, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₃ₐ, R₄ₐ and R₅ₐ are independently chosen from H, F, Cl, Br, I, —OH, —OR₇, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₆ₐ, R₇ₐ, R₈ₐ, R₉ₐ, R₁₀ₐ, R₁₁ₐ, R₁₂ₐ, R₁₃ₐ and R₁₄ₐ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl.

The expression “(C₁-C₁₂)alky/” refers to a linear or branched alkyl group comprising 1 to 12 carbon atoms.

With linear (C₁-C₁₂)alkyl group is meant methyl, ethyl, propyl, butyl, pentyl, hexyl, heptyl, octyl, nonyl, decyl, undecyl or dodecyl.

With branched (C₁-C₁₂)alkyl group is meant an alkyl group as defined above comprising substituents selected from the list of linear alkyl groups defined above, said linear alkyl groups being also capable of branching. Among the branched alkyl groups can be cited a tert-butyl, sec-butyl and isopropyl group.

A (C₁-C₁₂)alkyl group is in particular a (C₁-C₆)alkyl group, more in particular a (C₁-C₃)alkyl group and is preferably methyl or ethyl.

R₆, R₇, R₈, R₉, R₁₀, R₁₁ R₁₂, R₁₃ and R₁₄ are in particular independently chosen from (C₁-C₁₂)alkyl.

—CO₂R₆ is in particular a methyl ester or an ethyl ester.

—OR₇ is in particular methoxy or ethoxy.

—SR₈ is in particular —SCH₃.

—SOR₉ is in particular —SOCH₃.

—SO₂R₁₀ is in particular —SO₂CH₃.

—NHR₁₁ is in particular NHCH₃ or NHCH₂CH₃.

—N(R₁₂)₂ is in particular N(CH₃)₂ or N(CH₂CH₃)₂.

—NHCOR₁₃ is in particular —NHCO(CH₃).

—NHSO₂OR₁₄ is in particular NHSO₂OCH₃.

—NR₁₅ is in particular —NR₁₅ is chosen from —NCH₃, —NCH₃CH₃.

R₆ₐ, R₇ₐ, R₈ₐ, R₉ₐ, R₁₀ₐ, R₁₁ₐ R₁₂ₐ, R₁₃ₐ and R₁₄ₐ are in particular independently chosen from (C₁-C₁₂)alkyl.

—CO₂R₆ₐ is in particular a methyl ester or an ethyl ester.

—OR₇a is in particular methoxy or ethoxy.

—SR₈ₐ is in particular —SCH₃.

—SOR₉a is in particular —SOCH₃.

—SO₂R₁₀ₐ is in particular —SO₂CH₃.

—NHR₁₁ₐ is in particular NHCH₃ or NHCH₂CH₃.

—N(R₁₂ₐ)₂ is in particular N(CH₃)₂ or N(CH₂CH₃)₂. —NHCOR₁₃a is in particular —NHCO(CH₃).

—NHSO₂OR₁₄ₐ is in particular NHSO₂OCH₃.

The term “(C₁-C₁₂)haloalkyl” refers to a (C₁-C₁₂)alkyl as defined above, substituted by 1 or more halogen atoms, in particular chosen from F or C₁. —CF₃ is a particular example of (C₁-C₁₂)haloalkyl.

The term “(C₁-C₁₂)alkoxy” refers to (C₁-C₁₂)alkyl-O, wherein (C₁-C₁₂)alkyl is as defined above. Examples of “(C₁-C₁₂)alkoxy are methoxy and ethoxy.

The term “(C₂-C₁₂)alkenyl” refers to a (C₁-C₁₂)alkyl group as defined above, comprising 1 or more carbon-carbon double bonds. Examples of alkenyl include ethenyl or allyl.

The term “(C₂-C₁₂)alkynyl” refers to a (C₁-C₁₂)alkyl group as defined above, comprising 1 or more carbon-carbon triple bonds. Examples of alkynyl groups include ethynyl or propargyl.

The term “(C₁-C₁₂)heteroalkyl” refers to a (C₁-C₁₂)alkyl as defined above, further comprising, in its chain, a heteroatom, in particular chosen from 0, NH or S. As examples can be cited ethylene glycol groups (HO—CH₂—CH₂—O—).

The term “(C₃-C₁₂)cycloalkyl”, refers to a saturated cyclic carbon ring comprising 3 to 12 carbon atoms. The cycloalkyl is in particular a (C₃-C₆)cycloalkyl. Examples include cyclopropyl, cyclobutyl, cyclopentyl, and cyclohexyl.

The term “(C₃-C₁₂)heterocycloalkyl”, refers to a cycloalkyl as defined above, further comprising a heteroatom in its ring, in particular chosen from O or N. An example includes a tetrahydropyranyl ring.

The term “(C₆-C₁₈)aryl” refers to an aromatic group consisting of 6 to 18 carbon atoms in its ring. A (C₆-C₁₈)aryl group is in particular a (C₆-C₁₂)aryl group. Aryl is in particular phenyl.

A “(C₆-C₁₈)aryl-(C₁-C₁₂)alkyl” refers to a (C₁-C₁₂)alkyl as defined above, to which is attached a (C₆-C₁₈)aryl group as defined above. The (C₆-C₁₈)aryl-(C₁-C₁₂)alkyl group is in particular benzyl.

The term “heteroaryl” represents any monovalent radical of a monocyclic or polycyclic 5 to 10 membered aromatic group (such as a bicyclic group) comprising from 1 to 3 heteroatoms independently selected from oxygen, nitrogen and sulfur. The heteroaryl can optionally be substituted by one or more substituents, on a carbon- or heteroatom, said substituents being as defined above for “aryl”. Heteroaryl is in particular pyridyl, furanyl, imidazolyl, pyrrolyl.

A “(5 to 10 membered)heteroaryl-(C₁-C₁₂)alkyl” refers to a (C₁-C₁₂)alkyl as defined above, to which is attached an heteroaryl group as defined above.

The aryl or heteroaryl groups defined above may optionally be substituted by one or more substituents, in particular by 1 or 2 substituents, said substituents being in particular chosen from:
halogen, in particular F or Cl,
carboxylic ester, in particular methyl ester or ethyl ester,
(C₁-C₁₂)alkyl, in particular methyl, ethyl or iso-propyl,
(C₁-C₁₂)alkoxy, in particular methoxy or ethoxy,
carboxylic acid,
nitrile,
amine, in particular —NH₂, NHCH₃, or N(CH₃)₂.

In a particular embodiment, the aryl or heteroaryl groups are unsubstituted.

According to a particular embodiment, the ophthalmic lens according to the invention comprises a compound of Formula (I), said compound of Formula (I) not being in the form of a salt.

According to a particular embodiment, the ophthalmic lens according to the invention comprises a compound of Formula (I) in the form of a salt. This may notably be the case when said compound of Formula (I) comprises at least one basic nitrogen atom, capable of forming said salt, in particular wherein X is N or NR₁₅.

Alternatively, if the compound of Formula (I) comprises a carboxylic acid group, the salt of Formula (I) may be a carboxylate salt, in particular having a sodium, potassium or lithium counter-ion.

In particular, salts useful in the context of the present invention are hydrochloride salts or a sulfate salt.

According to a particular embodiment, R₁ and R₁ₐ are independently chosen from —CN or —CO₂Et.

According to a particular embodiment, R₂, R₂ₐ, R₃, R₃ₐ, R₄, R₄ₐ, R₅ and R₅ₐ are independently chosen from H or (C₁-C₁₂)alkyl, preferably H or CH₃.

According to a particular embodiment, R₂ and R₂ₐ are H.

According to a particular embodiment, R₄ and R₄ₐ are H.

According to a particular embodiment, R₃, R₃ₐ, R₅ and/or R₅ₐ is/are CH₃.

According to a particular embodiment, R₁₆ is chosen from halogen atoms, in particular chosen from F or Cl.

According to a particular embodiment, R₁₆ is chosen from (C₁-C₁₂)alkoxy, in particular chosen from methoxy or ethoxy.

According to a particular embodiment, R₁₆ is chosen from (C₆-C₁₈)aryl, in particular phenyl.

According to a particular embodiment, X is NH or NR₁₅,
R₁₅ is in particular —(C₁-C₁₂)alkyl, said alkyl being optionally substituted by one to three R₁₆ groups,
said R₁₆ being in particular chosen from F, methoxy or phenyl,
more in particular, —NR₁₅ is chosen from —NCH₃, —NCH₃CH₃—N—CH₂C(CH₂CH₃)(CH₂)₃CH₃, —NC(CH₃)₃, —NCH₂—CF₃, N(CH₂)₂-phenyl, N—(CH₂)₄—F or N—(CH₂)₄—OCH₃,
more in particular, X is NH or NCH₃.

According to a particular embodiment, the compound of Formula (I) is such that:
R₁ is —CN,
and
X is —NR₁₅,
R₁₅ being as defined in claim 1, in particular chosen from —(C₁-C₁₂)alkyl, optionally substituted by one to three R₁₆ groups, in particular chosen from halogen atoms, (C₁-C₁₂)alkoxy, (C₆-C₁₈)aryl,
or a compound of Formula a as defined in claim 1 in which R₁ₐ is —CN, and R₂ₐ, R₃ₐ, R₄ₐ and R₅ₐ are H.

According to a particular embodiment the compound of Formula (I) is such that:
R₁ is —CN,
R₂, R₃, R₄ and R₅ are H, and
X is —NR₁₅,
R₁₅ being as defined above, in particular chosen from —(C₁-C₁₂)alkyl, optionally substituted by one to three R₁₆ groups, in particular chosen from halogen atoms, (C₁-C₁₂)alkoxy, (C₆-C₁₈)aryl,
or a compound of Formula a as defined above in which R₁ₐ is —CN, and R₂ₐ, R₃ₐ, R₄ₐ and R₅ₐ are H.

The inventors have surprisingly found that when X is NR₁₅, the ophthalmic lens as disclosed herein is particularly stable upon long-term storage. In particular, such lenses when stored for more than one year, retain adequate optical properties.

According to a particular embodiment R₁ₐ is equal to R₁, R₂ₐ is equal to R₂, R₃ₐ is equal to R₃ and R₄ₐ is equal to R₄.

According to a particular embodiment, the compound of Formula (I) is such that when R₂ is a (C₆-C₁₈)aryl or a (5 to 10)membered heteroaryl, R₁ is —CONH₂ or —CO₂R₆, R₆ being as defined above.

In a particular embodiment of formula (I), R₂ is chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl-(C₁-C₁₂)alkyl, or (5 to 10 membered)heteroaryl-(C₁-C₁₂)alkyl.

In a particular embodiment, L is chosen from propylene or butylene, in particular propylene.

According to a particular embodiment, the compound of Formula (I) has the structure of Formula (II), (III), (IV), (V), in particular of Formula (II):

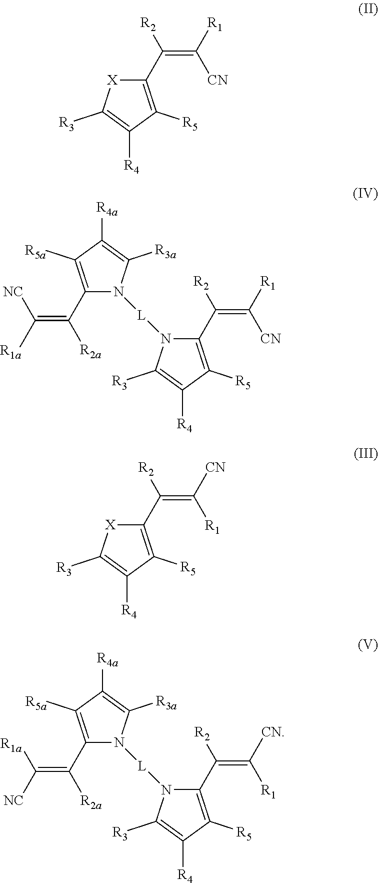

If R₁ is —CN, compounds (II) and (III) are structurally the same. If R₁ is —CO₂R₆ or —CO₂NH₂, compounds (II) and (III) are different with respect to the stereochemistry of the double bond, which can be in the Z or in the E configuration. In compound (I), the stereochemistry might be such that a mixture of E and Z double bonds exists.

According to a particular embodiment, when R⁶ is (C₂-C₁₂)alkenyl, then X is S, NH or —NR¹⁵ as defined above.

In an embodiment, R¹, R^{1a}, R², R^{2a}, R³, R^{3a}, R⁴, R^{4a}, R⁵, R^{5a}, R^{6a} and R⁶ are not (C₂-C₁₂)alkenyl.

According to a particular embodiment, the compound of Formula (I) is chosen from Formulae (I-1), (I-2), (I-3), (I-4), (I-5), (I-6) or (I-7):

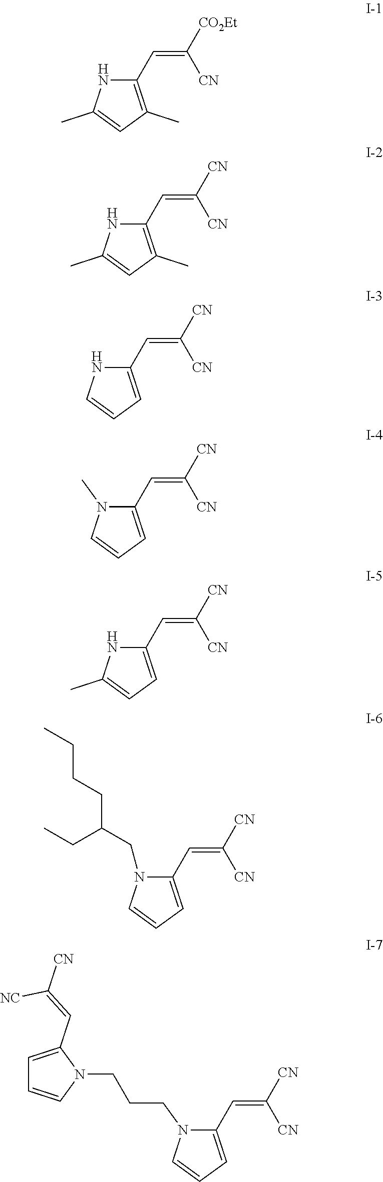

In particular, the compound of Formula (I) is comprised in the ophthalmic lens as such, meaning that the compound of Formula (I) is not covalently bound to the plastic base.

The compound of Formula (I) comprised in the ophthalmic lens of the invention may exhibit a significant absorption in the range of blue light. In particular, the compound of Formula (I) may have a maximum absorption wavelength (λₘₐₓ) lower than or equal to 400 nm. More particularly, the compound of Formula (I) may have a maximum absorption wavelength in the range of from 350 to 400 nm, in particular of from 360 to 400 nm, in particular of from 370 to 400 nm, more in particular of from 370 to 395 nm.

A maximum absorption wavelength above these ranges might lead to an undesirable high yellow index of the lens in which said compound of Formula (I) is comprised. A maximum absorption wavelength below these ranges may lead to insufficient absorption in the blue light range.

At the maximum absorption wavelength (λₘₐₓ), the compound of Formula (I) may have a molar absorption extinction coefficient (ε) equal to or higher than 30,000 M⁻¹cm⁻¹.

More particularly, the compound of Formula (I) may have a molar absorption extinction coefficient (ε) in the range of from 30,000 to 80,000 M⁻¹cm⁻¹, more in particular in the range of from 30,000 to 70,000 M⁻¹cm⁻¹, even more in particular in the range of from 30,000 to 45,000 M⁻¹cm⁻¹.

The λₘₐₓ and ε of the compound of Formula (I) may be measured on a 20 ppm solution of the compound of Formula (I) in an organic solvent such as ethanol, acetonitrile or tetrahydrofuran, in particular in tetrahyrofuran.

The “molar absorption extinction coefficient” is a measurement of how strongly a chemical species absorbs, and thereby attenuates, light at a given wavelength. It is an intrinsic property of the species. It is defined by the ratio of absorption to the length of the optical path moved by electromagnetic radiation in a given solution.

This is expressed as M⁻¹m⁻¹. It does not depend on the concentration of the solution nor on the thickness crossed by the light; on the other hand, it depends on the wavelength of the incident light and on the nature of the solute and the solvent.

The compound of Formula (I) may be obtained by a Knoevenagel condensation according to the following scheme:

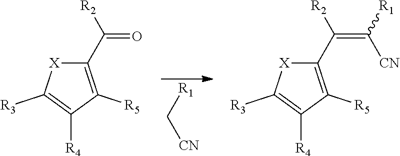

After the reaction is complete, the crude product may be purified by known techniques, such as crystallization or column chromatography.

The ophthalmic lens of the invention further comprises a plastic base.

As used herein, the term “plastic base” refers to a bare ophthalmic substrate such as an unfinished, untreated, or uncoated ophthalmic lens.

Preferably, the plastic base of the ophthalmic lens of the invention is transparent.

As used herein, the term “transparent” is intended to mean a material having a transmission greater than 85%, preferably greater than or equal to 90%.

The refractive index of the plastic base may be in the range from about 1.4 to about 1.9, such as about 1.5 to about 1.9, about 1.6 to about 1.9, about 1.7 to about 1.9, about 1.8 to about 1.9, about 1.4 to about 1.8, about 1.4 to about 1.7, about 1.4 to about 1.6, about 1.45 to about 1.85, about 1.45 to about 1.75, or about 1.5 to about 1.7, in particular from 1.49 to 1.74.

The plastic base of the ophthalmic lens of the invention may be any conventional optical substrate known in the art. In particular, the plastic base may be chosen from a thermoset resin or a thermoplastic resin, in particular a thermoset resin. The resin may be chosen from resins such as a polyamide, polyimide, polysulfone, polycarbonate, polyethylene terephthalate, poly(methyl(meth)acrylate), cellulose triacetate or copolymers thereof, or is chosen from a thermosetting resin, such as a cyclic olefin copolymer, a homopolymer or copolymer of allyl esters, a homopolymer or copolymer of allyl carbonates of linear or branched aliphatic or aromatic polyols, a homopolymer or copolymer of (meth)acrylic acid and esters thereof, a homopolymer or copolymer of thio(meth)acrylic acid and esters thereof, a homopolymer or copolymer of urethane and thiourethane, a homopolymer or copolymer of epoxy, a homopolymer or copolymer of sulphide, a homopolymer or copolymer of disulphide, a homopolymer or copolymer of episulfide, a copolymer of polythiol and polyisocyanate, and combinations thereof.

In one embodiment, the plastic base may comprise a copolymer of at least one polythiol and a polyisocyanate; preferably a copolymer of xylylene diisocyanate and 1,2-bis[(2-mercaptoethyl)thio]-3-mercaptopropane or a copolymer of bis(isocyanatomethyl)bicyclo[2.2.1]heptane, pentaerythritol tetrakis(3-mercaptopropionate) and 1,2-bis[(2-mercaptoethyl)thio]-3-mercaptopropane or a copolymer of xylylene diisocyanate and 4,8 (or 4,7 or 5,7)-dimercaptomethyl-1,11-dimercapto-3,6,9-trithiaundecane.

In another embodiment, the plastic base may be a copolymer of an allyl monomer or allyl oligomer, such as diethylene glycol bis(allyl carbonate), ethylene glycol bis(allyl carbonate), oligomers of diethylene glycol bis(allyl carbonate), oligomers of ethylene glycol bis(allyl carbonate), bisphenol A bis(allyl carbonate) or diallylphthalates, and a second monomer or oligomer that is capable of polymerizing with the allyl monomer or oligomer, such as aromatic vinyl compounds alkyl mono(meth)acrylates, di(meth)acrylates, tri(meth)acrylates or tetra(meth)acrylates.

In the ophthalmic lens of the invention, the compound of Formula (I) may be included in the plastic base or may be included in a separate layer coated on a surface of the plastic base.

As used herein, the separate layer coated on a surface of the plastic base is defined as a coating, which (i) is positioned above the substrate, (ii) is not necessarily in contact with the substrate, that is to say one or more intermediate layers may be arranged between the substrate and the layer in question, and (iii) does not necessarily completely cover the substrate.

The separate layer coated on a surface of the plastic base may be made of any material conventionally used to coat ophthalmic lens. For example, said separate layer may be obtained by polymerization of a (meth)acrylic based coating or a sol-gel based coating or an epoxy based coating or polyurethane based coating.

Preferably, the compound of Formula (I) is included in the plastic base of the ophthalmic lens. The compound of Formula (I) may be included in the plastic base by dispersion of compound of Formula (I) in raw materials (monomers) before polymerization. As such, the compound of Formula (I) may be homogeneously dispersed within the plastic base of the ophthalmic lens. Alternatively, the compound of Formula (I) may be included in the plastic base by imbibition. A method for imbibition may comprise soaking the plastic base in a bath containing the compound of Formula (I) for a determined time, so that the compound of Formula (I) may diffuse into the plastic base. As such, the compound of Formula (I) may be dispersed into a thin layer at a surface of the plastic base. With imbibition method, the thickness of the plastic base which includes compound of Formula (I) does not depend on the global geometry of the ophthalmic lens.

When the compound of Formula (I) is included in the plastic base, the amount of compound of Formula (I) may be comprised in the range of from 0.001 to 2%, in particular of from 0.001 to 1%, more in particular of from 0.001 to 0.1%, even more in particular of from 0.01 to 0.05% or of from 0.003 to 0.035%, by weight based on the weight of the plastic base.

When the compound of Formula (I) is included in a separate layer coated on a surface of the plastic base, the amount of compound of Formula (I) may be comprised in the range of from 0.001 to 2%, in particular of from 0.001 to 1%, more in particular in the range of from 0.001 to 0.1%, even more in particular in the range of from 0.01 to 0.05% or of from 0.003 to 0.035%, by weight based on the weight of the separate layer.

Further, the compound of Formula (I) may be encapsulated within nanoparticles. Said nanoparticles may be dispersed within the plastic base of the ophthalmic lens or may be dispersed within a separate layer coated on a surface of the plastic base.

Preferably, said nanoparticles are dispersed within the plastic base of the ophthalmic lens, said plastic base comprising a copolymer including an allyl monomer or allyl oligomer as defined above.

The amount of compound of Formula (I) in the nanoparticles may be comprised in the range of from 0.0001 to 90 wt %, in particular in the range of from 0.01 to 50% wt %, more particularly in the range of from 0.1 to 10 wt % based on the weight of the nanoparticles.

In the context of the present invention, the term “nanoparticles” is intended to mean individualized particles of any shape having a size, measured in its longest direction, in the range of 1 nm to 10 μm, preferably in the range of 10 nm to 5 μm, as measured by Dynamic Light Scattering.

The nanoparticles may be either polymer-based, i.e. they comprise a polymer, or mineral-based, i.e. they comprise a mineral oxide.

In a preferred embodiment, the polymer or mineral oxide comprised in the nanoparticles is a transparent material.

The ophthalmic lens may further comprise a UV-absorber other than the compound of Formula (I). In one embodiment, the ophthalmic lens may further comprise a benzotriazole UV-absorber or a mixture thereof, preferably a benzotriazole UV-absorber selected from 2-(2-hydroxy-5-tert-octylphenyl)-2H-benzotriazole, 2-(3-tert-butyl-2-hydroxy-5-methylphenyl)-5-chloro-2H-benzotriazole, and mixtures thereof.

The benzotriazole UV-absorber may be included in the plastic base or may be included in a separate layer coated on a surface of the plastic base.

Preferably, the benzotriazole UV-absorber is included in the plastic base when the compound of Formula (I) is included in the plastic base or the benzotriazole UV-absorber is included in a separate layer when the compound of Formula (I) is included in a separate layer coated on a surface of the plastic base.

Advantageously, the amount of benzotriazole UV-absorber is lower than that is generally necessary to absorb blue light when no compound of Formula (I) is present in the ophthalmic lens. When the benzotriazole UV-absorber is included in the plastic base, the total amount of benzotriazole UV-absorber may be comprised in the range of from 0.001 to 2%, in particular in the range of from 0.01 to 1.5%, more particularly in the range of from 0.1 to 1.25%, by weight based on the weight of the plastic base. When the benzotriazole UV-absorber is included in a separate layer coated on a surface of the plastic base, the total amount of benzotriazole UV-absorber may be in the range of from 0.001 to 2%, in particular in the range of from 0.01 to 1.5%, more particularly 0.1 to 1.25%, by weight based on the weight of the separate layer.

The ophthalmic lens may further comprise an additive, in particular a light stabilizer, or an anti-yellowing agent. As used herein, “light stabilizer” is intended to mean an additive that improves light stability of the ophthalmic lens, in particular an additive that limits degradation of the ophthalmic lens properties upon exposure to light, such as ultra-violet light. As used herein, “anti-yellowing agent” is intended to mean an additive that limits yellowing of the ophthalmic lens. Particularly suitable light stabilizers are sulfide anti-yellowing additive such as dialkyl thiodipropionate or dialkyl thiodiglycolate and hindered amine light stabilizer (HALS) such as Tinuvin® 144, Tinuvin® 1130, Tinuvin® 479, Tinuvin® 123 or Hostavin® 3058, or a mixture thereof.

The light stabilizer may be included in the plastic base or may be included in a separate layer coated on a surface of the plastic base.

Preferably, the light stabilizer is included in the plastic base when the compound of Formula (I) is included in the plastic base, or the light stabilizer is included in a separate layer when the compound of Formula (I) is included in a separate layer coated on a surface of the plastic base.

Advantageously, the amount of light stabilizer is comprised in the range of from 0.001 to 1%, in particular in the range of from 0.01 to 0.75%, more particularly in the range of from 0.025 to 0.5%, by weight based on the weight of the plastic base or of the separate layer coated on a surface of the plastic base.

Use of light stabilizer is particularly desirable when the plastic base is prone to yellowing, in particular for a homopolymer or copolymer of allyl carbonates of linear or branched aliphatic or aromatic polyols; or a copolymer of polythiol and polyisocyanate. For the latter (polythiourethanes), use of light stabilizer is desirable for plastic bases with high refractive index around 1.6, and more particularly around 1.67.

As mentioned above, the ophthalmic lens may be manufactured in accordance with wearer specifications and may be processed to provide the ophthalmic lens with various functions. Accordingly, the ophthalmic lens may have a complex structure resulting from interlayering of materials and/or a series of treatments to tailor the ophthalmic lens to specific user requirements. For example, the treatments may be carried out to reduce thickness and to render the ophthalmic lens lightweight, to improve its transparency, for durability, strength and protection, aesthetics etc. It follows that an ophthalmic lens may further comprise one or more coatings disposed on the plastic base in addition to the optional separate layer that may comprise the compound of Formula (I), such as an impact resistant coating, an anti-scratch coating, an anti-reflection coating, a tint coating, a color coating, an anti-static coating, an anti-smudge coating, a water repellent coating, a polarizing coating, or a photochromic coating.

The ophthalmic lens of the invention may absorb blue light. In particular, the ophthalmic lens may exhibit light cut higher than 403 nm.

In one embodiment, the ophthalmic lens may exhibit light cut comprised in the range of from 403 to 420 nm, in particular in the range of from 404 to 411 nm.

The term “light cut”, or optical cut (UVₒᵤₜ), as used herein refers to the lowest wavelength in UV-visible range for which transmittance through the ophthalmic lens is greater than 1%.

The term “transmittance” as used herein refers to intensity of radiation transmitted through a material over that of the incident radiation, and which is expressed as a percentage. The transmittance of the ophthalmic lens can be measured according to ISO 8980-3-2003.

In one embodiment, the transmittance TVD65 of the of the ophthalmic lens is greater than 85%, in particular greater than 87%.

The ophthalmic lens of the invention may not exhibit undesirable yellowing. In particular, the Yellow Index, or YI, of light transmitted through said ophthalmic lens may be lower than 14. The Yellow Index of the ophthalmic lens can be measured according to ASTM D-1925.

In particular, the Yellow Index of light transmitted through said ophthalmic lens may be lower than 10 at a UV_{cut} of 410 nm.

The ophthalmic lens of the invention may have a BVC B′ value is greater than 25%, in particular greater than 30%, more in particular greater than 35%.

The BVC B′ value refers to the Blue violet cut, determined by excluding transmission in range of 400-455 nm from the full transmission in range of 380-780 nm (100%−Mean transmittance in the spectral range 400-455 nm).

The ophthalmic lens according to the invention exhibits good stability.

The ophthalmic lens as described above, may have an optical cut change ΔUV_{cut} after exposure of the ophthalmic lens for 40 hours to light in a Q-SUN Xe-3 xenon test chamber of less than 2%, in particular of less than 1%, these values being absolute values.

The ophthalmic lens as described above, may have a perceived color difference ΔE_{lab} after exposure of the ophthalmic lens for 40 hours to light in a Q-SUN Xe-3 xenon test chamber of less than 2.5, in particular of less than 1.

ΔUV_{cut} (%) denotes the optical cut change after accelerated aging for 40 hours (t40) in a Q-SUN Xe-3 xenon test chamber relative to lens at the initial state (t0), expressed in in percentage:

$$Δ {UV}_{cut} ( \% ) = \frac{{UV}_{cut ( t 40 )} - {UV}_{cut ( t 0 )}}{{UV}_{cut ( t 0 )}} \times 100$$

ΔE_{lab}, denoting perceived color difference after accelerated aging for 40 hours (t40) in a Q-SUN Xe-3 xenon test chamber relative to lens at the initial state (t0):

$$Δ {E}_{L a b} = \sqrt{{( Δ {L}^{*} )}^{2} + {( Δ {a}^{*} )}^{2} + {( Δ {b}^{*} )}^{2}}$$

In which each individual differences stands for:

$$Δ {L}^{*} = {L}_{t 40}^{*} - {L}_{t 0}^{*}$$

$$Δ {a}^{*} = {a}_{t 40}^{*} - {a}_{t 0}^{*}$$

$$Δ {b}^{*} = {b}_{t 40}^{*} - {b}_{t 0}^{*}$$

L* corresponding to lightness
a* value indicating red-green component
b* value indicating yellow-blue components

These values Colorimetric coordinates L*, a* and b*, represent colorimetric coordinates that can be measured according to the international colorimetric system CIE (1976) L*a*b*.

In addition, and in particular, it has been found that when X in Formula (I) is NR₁₅, the ophthalmic lens is particularly stable in the long term.

Such lenses still show adequate optical properties after at least one year of storage in the dark and at room temperature. They notably display after one year of storage:
an optical cut change ΔUV_{cut} of less than 0.5%, these values being absolute values, and/or
a perceived color difference ΔEa_{b} of less than 1, and/or
a Δ BVC B′ value of less than 5%.

### Process for the Preparation of the Ophthalmic Lens

The ophthalmic lens of the invention may be prepared with a process comprising the steps of:
a) providing monomers or oligomers from which the plastic base can be prepared;
b) mixing the monomers or oligomers, the compound of Formula (I) and a catalyst suitable for the polymerization of the monomers and oligomers to form a polymerizable liquid composition;
c) curing the polymerizable liquid composition.

A coating may be deposited or formed through various methods, including wet processing, and film transfer.

In particular, the ophthalmic lens of the invention may be prepared with a wet process comprising the steps of:
a) providing monomers or oligomers from which a coating layer can be prepared;
b) mixing the monomers or oligomers, the compound of Formula (I) and a catalyst suitable for the polymerization of the monomers and oligomers to form a polymerizable liquid composition;
c) coating the polymerizable liquid composition on a substrate;
d) curing the polymerizable liquid composition.

Preferably, the curing is a thermal curing.

Alternatively, the ophthalmic lens of the invention may be prepared with a film transfer process comprising the steps of:
a) providing monomers or oligomers from which a coating layer can be prepared;
b) mixing the monomers or oligomers, the compound of Formula (I) and a catalyst suitable for the polymerization of the monomers and oligomers to form a polymerizable liquid composition;
c) coating the polymerizable liquid composition on a transfer film;
d) curing the polymerizable liquid composition to obtain a coating layer;
e) transferring the coating layer from the transfer film onto a substrate.

Preferably, the curing is a thermal curing.

Preferably, the coating layer is glued to the substrate by an adhesive layer, such as a UV curable adhesive or a thermally curable adhesive layer or a pressure sensitive adhesive layer.

According to a preferred embodiment, the polymerizable liquid composition may be stirred until homogeneous and subsequently degassed and/or filtered before curing.

The polymerizable liquid composition of the present invention described above may be cast into a casting mold for forming a lens and polymerized by heating at a temperature of from 10 to 150° C., in particular from 15 to 130° C. According to a preferred embodiment, the heating may last for 5 to 24 hours, preferably 7 to 22 hours, more preferably 15 to 20 hours.

The casting mold may then be disassembled and the lens may be cleaned with water, ethanol or isopropanol.

In the process according to the invention, a mold releasing agent may be used. A mold releasing agent prevents excessive adhesion between lens and the casting mold, through creation of barrier between lens and the casting mold surface. Both internal and external mold releasing agent may be used in the invention.
An internal mold releasing agent is used by adding such said agent in the monomer bulk, for example during step b) of the process according to the invention. As non-limiting examples of an internal mold releasing agent can be cited mono- or di-alkyl phosphate, acidic mono- or di-phosphate esters, silicone, fluorinated hydrocarbon, fatty acids, and ammonium salts. When an internal mold releasing agent is used, the agent may be present in the prepared ophthalmic lens.
An external mold releasing agent is used by coating inner surface of casting mold with a surface lubricant such as siloxane and fluorinated hydrocarbon.

The ophthalmic lens may then be coated with one or more functional coatings selected from the group consisting of an impact resistant coating, an anti-scratch coating, an anti-reflection coating, a tint coating, a color coating, an anti-static coating, an anti-smudge coating, a water repellent coating, a polarizing coating, or a photochromic coating.

### Use of a Compound of Formula (I)

The present invention also pertains to the use of a compound of Formula (I) as defined above in an ophthalmic lens to absorb blue light.

The absorption of blue light can be assessed by determining the average transmittance of the ophthalmic lens over the range 420-450 nm. Advantageously, the average transmittance of the ophthalmic lens over the range 420-450 nm is less than 85%, for an uncoated lens (i.e. without anti-reflection coating).

### Composition Comprising an Acrylonitrile Compound

The present invention also pertains to a composition comprising a plastic base and a compound of Formula (IA):

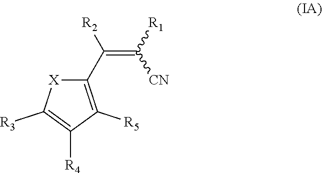

wherein:
wherein:
R₁ is chosen from —CN, —CONH₂, —CO₂R₆, or —SO₂—(C₁-C₁₂)alkyl R₂ is, independently from R₃, R₄ and R₅, chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, or (5 to 10 membered)heteroaryl-(C₁-C₁₂)alkyl,
R₃, R₄ and R₅ are independently chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl, X is NH, NR₁₅, O, or S,
R₆, R₇, R₈, R₉, R₁₀, R₁₁ R₁₂, R₁₃ and R₁₄ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl, R₁₅ is chosen from (C₁-C₁₂)alkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl, (5 to 10 membered)heteroaryl-(C₁-C₂)alkyl, said alkyl being optionally substituted by one to three R₁₆ groups,
R₁₆ is chosen from halogen atoms, (C₁-C₁₂)alkoxy, (C₆-C₁₈)aryl,
or wherein R₁₅ is or a structure of Formula b:

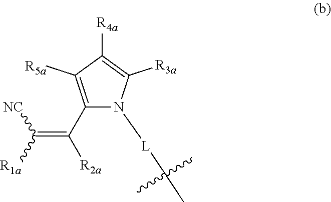

in which
L is (C₁-C₁₂)alkylene,
R₁ₐ is chosen from —CN, —CONH₂, —CO₂R₆ₐ or —SO₂—(C₁-C₁₂)alkyl,
R₂ₐ is, independently from R₃ₐ, R₄ₐ and R₅ₐ, chosen from H, F, Cl, Br, I, —OH, —OR₇ₐ, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₃ₐ, R₄ₐ and R₅ₐ are independently chosen from H, F, Cl, Br, I, —OH, —OR₇, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₆ₐ, R₇ₐ, R₈ₐ, R₉ₐ, R₁₀ₐ, R₁₁ₐ, R₁₂ₐ, R₁₃ₐ and R₁₄ₐ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl,
wherein the aryl or heteroaryl groups are optionally substituted by one or more substituents, in particular by 1 or 2 substituents, said substituents being in particular chosen from:
halogen, in particular F or C₁,
carboxylic ester, in particular methyl ester or ethyl ester,
(C₁-C₁₂)alkyl, in particular methyl, ethyl or iso-propyl,
(C₁-C₁₂)alkoxy, in particular methoxy or ethoxy,
carboxylic acid,
nitrile,
amine, in particular —NH₂, NHCH₃, or N(CH₃)₂,

wherein the plastic base is as defined above.

The present invention also pertains to the use of a composition as defined above in an optical article.

The use according to the invention in particular pertains to optical articles such as optical lenses, display devices, optical filters, window panes, protective adhesives and films, in particular ophthalmic lenses, or display devices such as computer screens or tv screens.

In particular, the composition according to the invention is used in said optical article to absorb blue light.

The present invention further relates to a novel compound chosen from:

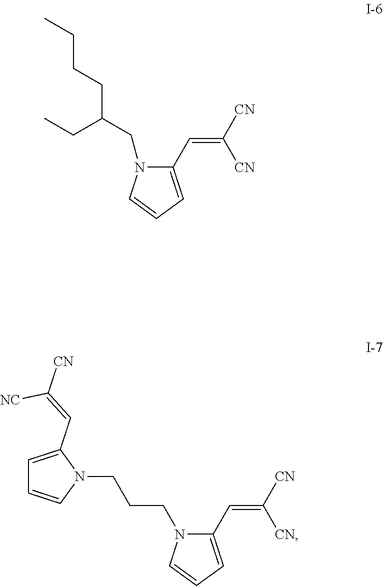 [SMILES: CCCCC(CC)Cn1cccc1C=C(C#N)C#N ; N#CC(C#N)=Cc1cccn1CCCn1cccc1C=C(C#N)C#N]

or salts thereof.

The invention will now be described in more detail with the following examples which are given for purely illustrative purpose and which are not intended to limit the scope of the invention in any manner.

The invention will now be described in more detail with the following FIGURE and examples which are given for purely illustrative purposes, and which are not intended to limit the scope of the invention in any manner.

### FIGURE

FIG. 1 represents a graph of absorbance (A) as a function of wavelength (λ, in nm) for a solution of 20 ppm of compound of Formulae (I-1) to (I-7) in tetrahydrofuran.

### EXAMPLES

### Measuring Methods for the Optical Performance and Light Resistance of the Lenses.

Optical properties of lenses were measured on plano lenses with 2 mm center thickness using a Cary60 UV Vis spectrophotometer spectrometer, using a scan wavelength of 280-780 nm and photo-aging test were performed on Q-sun (40 h, light power 0.68 W/m²). The optical values include UV_{cut}, TvD65, Yl, C*, h*, a*, b* and BVC B′.
UV_{cut} is determined under normal incident light by plotting the graph of the transmittance percentage of the lens as a function of the wavelength. The UV_{cut} of the material can be read on the graph as the lowest wavelength from the spectrum for which transmittance through the ophthalmic lens is greater than 1%.
TVD65 is determined under normal incident light (standard illuminant D65).
BVC B′ is determined by excluding transmission in range of 400-455 nm from the full transmission in range of 380-780 nm (100%−mean transmittance in the spectral range 400-455 nm)
YI is measured according to ASTM D1925.
Colorimetric coordinates L*, a*, b*, C* and h* of the lenses of the invention were measured according to the international colorimetric system CIE (1976) L*a*b* and L*C*h* international colorimetric system with D65 source and 10° observer; The measured parameters are:
L* corresponding to lightness
a* value indicating red-green component
b* value indicating yellow-blue components
C*, the chroma, representing saturation of the color
h* representing the angle on the chromaticity axes.

Light resistance by Q-sun test were performed on piano lenses with 2 mm center thickness, and were performed on Q-sun Xe-1 model: 0.68 W/m2 of light intensity at 340 nm, temperature at 60° C., daylight Q-UV filter, and 40 h of running cycle. The light resistance of lenses was qualified in two main aspects: lens optical cut change (ΔUV_{cut}) and color evolution (ΔE_{lab}). Values were obtained by comparing the values measured after manufacture (t0) and the values after accelerated aging for 40 hours (t40) in the Q-sun cell.

### Materials

In the examples, the following compounds are used:

| Function | Chemical | CAS No. | Supplier |
|---|---|---|---|
| Monomer ISO1 | bis(isocyanatomethyl) | 74091-64-8 | Mitsui |
| (diisocyanate) | bicyclo[2.2.1]heptane | | Chemicals |
| Monomer THIO1 | pentaerythritol tetrakis(3- | 7575-23-7 | Mitsui |
| (polythiol) | mercaptopropionate) | | Chemicals |
| Monomer | 1,2-bis[(2- | 131538-00-6 | Mitsui |
| THIO2(polythiol) | mercaptoethyl)thio]- | | Chemicals |
| | 3-mercaptopropane | | |
| Monomer ISO2 | xylylene diisocyanate | 3634-83-1 | Mitsui |
| (diisocyanate) | | | Chemicals |
| Catalyst | Dimethyltin dichloride | 753-73-1 | Honjo |
| | (DMC) | | Chemical |
| Mold release | Zelec ® UN | — | Stepan |
| agent | | | Company |
| UV Absorber | Seesorb ® 703 | 3896-11-6 | Shipro |
| (benzotriazole) | | | Kasei |
| | | | Kaisha |
| UV Absorber | Seesorb ® 709 | 3147-75-9 | SC |
| (benzotriazole) | | | Organic |
| | | | Chemical |
| | | | Co., Ltd |
| UV Absorber | Seesorb ® 701 | 2440-22-4 | Shipro |
| (benzotriazole) | | | Kasei |
| | | | Kaisha |
| UV Absorber | Hippuric-benzaldehyde | 17606-70-1 | Eurofins |
| (H-Oxa) | azalactone | | |
| UV Absorber | Tinuvin ® 928 | 73936-91-1 | BASF |
| (benzotriazole) | | | |
| | 3,5-Dimethyl-1H-pyrrole-2- | 2199-58-8 | |
| | carbaldehyde | | |
| | pyrrole-2-carboxaldehyde | 1003-29-8 | |
| | 1-Methyl-1H-pyrrole-2- | 1192-58-1 | |
| | carboxaldehyde | | |
| | 5-Methyl-1H-pyrrole-2- | 1192-79-6 | |
| | carboxaldehyde | | |
| | Ethyl cyanoacetate | 105-56-6 | |
| | malonitrile | 109-77-3 | |
| | 2-ethylhexyl bromide | 18908-66-2 | |
| | 1,3-dibromopropane | 109-64-8 | |

### Example 1: Preparation of Compounds (I-1)-(I-7)

The following compounds (I-1)-(I-7) were prepared by a Knoevenagel condensation.

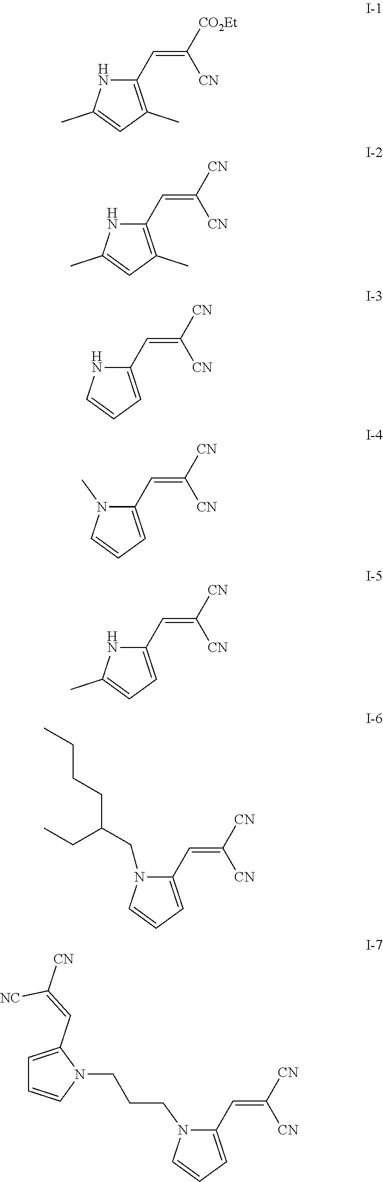

### Preparation of I-1

Compound I-1 was prepared according to the following reaction scheme:

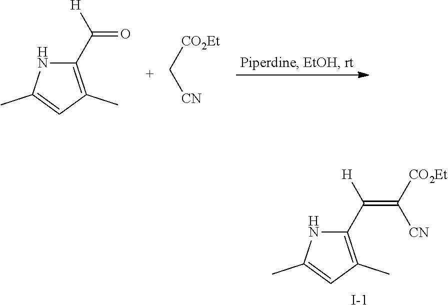

Briefly, 3,5-Dimethyl-1H-pyrrole-2-carbaldehyde was reacted with ethyl cyanoacetate in ethanol at room temperature, in the presence of piperidine.

### Preparation of I-2

Compound I-2 was prepared using the same reaction conditions as for compound I-1 but replacing the reactant ethyl cyanoacetate with malononitrile.

### Preparation of I-3

Compound I-3 was prepared according to the following reaction scheme:

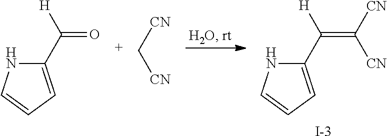

Briefly, pyrrole-2-carboxaldehyde was reacted with malonitrile in water at room temperature.

### Preparation of I-4

Compound I-4 was prepared using the same reaction conditions as for compound I-3 but replacing the reactant pyrrole-2-carboxaldehyde with 1-Methyl-1H-pyrrole-2-carboxaldehyde.

### Preparation of I-5

Compound I-5 was prepared using the same reaction conditions as for compound I-3 but replacing the reactant pyrrole-2-carboxaldehyde with 5-Methyl-1H-pyrrole-2-carboxaldehyde.

### Preparation of I-6

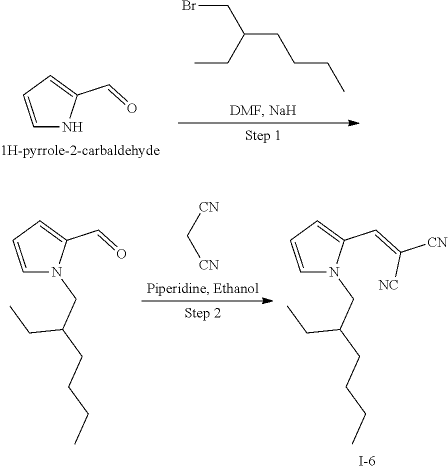

Compound I-6 was prepared using the same reaction conditions as for compound I-3 but replacing the reactant pyrrole-2-carboxaldehyde with 1-(2-ethylhexyl)-1H-pyrrole-2-carboxaldehyde. The latter was prepared by alkylating 1H-pyrrole-2-carbaldehyde with 2-ethylhexyl bromide in dimethylformamide, in the presence of sodium hydride.

### Preparation of I-7

Compound I-7 was prepared by alkylating pyrrole-2-carboxaldehyde with 1,3-dibromopropane, resulting in substitution of the 2 bromide groups. The 2 aldehyde groups were then reacted using the same reaction conditions as for compound I-3, to form the compound of Formula I-7:

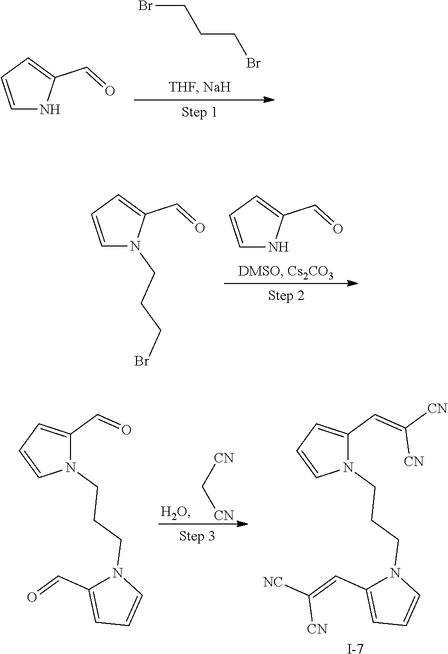

### Example 2: Absorption Properties of Compounds (I-1)-(1-7)

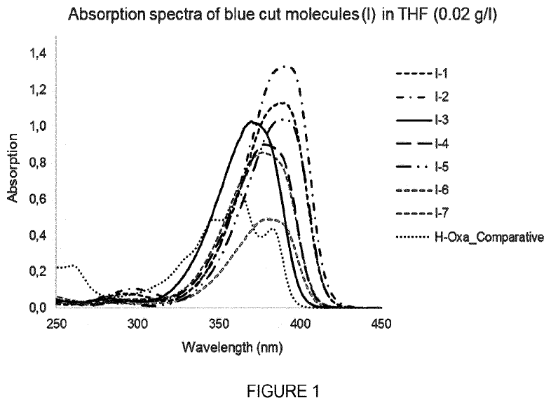

The absorbance (A) as a function of wavelength (λ) was measured for a solution of 0.02 mg of compound of formula (I-1) to (I-7) in 1 mL of tetrahydrofuran. The resulting absorption spectra are shown in FIG. 1.

The maximum absorption wavelength (λₘₐₓ) and the absorption extinction coefficients (ε) at the maximum absorption wavelength is given in the Table 1 below, for each compound:

TABLE 1
Absorption data of the inventive compounds

Absorption-THF

| | λₘₐₓ (nm) | ε (at λₘₐₓ) >30,000 |
|---|---|---|
| Compound | 360-400 nm | M⁻¹cm⁻¹ |
| I-1 | 389 | 40954 |
| I-2 | 392 | 37821 |
| I-3 | 370 | 37034 |
| I-4 | 378 | 35393 |
| I-5 | 391 | 41282 |
| I-6 | 379 | 31155 |
| I-7 | 377 | 69759 |
| H-Oxa: comparative | 383 | 26788 |
| Seesorb ® 703: | 353 | 17500 |
| Benzotriazole comparative | | |

All compounds according to the invention exhibit a maximum absorption wavelength (λₘₐₓ) in the range of 360 to 400 nm. In addition, the compounds according to the invention show greater extinction coefficients at the maximum absorption wavelength than the comparative prior art compounds H-Oxa and Seesorb® 703.

### Example 3: Preparation of Compositions to Obtain Ophthalmic Lenses with Refractive Index 1.67

Compositions 1 to 8 according to the invention and comparative compositions Comp. 1 to 3, comprising the following ingredients were prepared. The values expressed in Table 2 are weight percentages based on the total weight of the compositions.

TABLE 2
Compositions for ophthalmic lenses with refractive index 1.67.

| | Examples according to the invention | | | | | | | | Comparative examples | | |
|---|---|---|---|---|---|---|---|---|---|---|---|
| Composition (%) | 1 | 2 | 3 | 4 | 5 | 6 | 7 | 8 | Comp. 1 | Comp. 2 | Comp. 3 |
| ISO2 | 51.389 | 51.390 | 51.387 | 51.385 | 51.388 | 51.390 | 51.386 | 51.386 | 51.376 | 51.371 | 51.061 |
| THIO2 | 47.284 | 47.285 | 47.283 | 47.281 | 47.284 | 47.285 | 47.282 | 47.282 | 47.272 | 47.268 | 46.983 |
| Stan DMC | 0.010 | 0.010 | 0.010 | 0.010 | 0.010 | 0.010 | 0.010 | 0.010 | 0.010 | 0.010 | 0.010 |
| Zelec ® UN | 0.079 | 0.079 | 0.079 | 0.079 | 0.079 | 0.079 | 0.079 | 0.079 | 0.079 | 0.079 | 0.078 |
| Seesorb ® 701P-UV absorber | 1.233 | 1.233 | 1.233 | 1.233 | 1.233 | 1.233 | 1.233 | 1.233 | 1.233 | 1.233 | |
| I-1 | 0.004 | | | | | | | | | | |
| I-2 | | 0.002 | | | | | | | | | |
| I-3 | | | 0.008 | 0.012 | | | | | | | |
| I-4 | | | | | 0.006 | | | | | | |
| I-5 | | | | | | 0.003 | | | | | |
| I-6 | | | | | | | 0.010 | | | | |
| I-7 | | | | | | | | 0.010 | | | |
| H-Oxa | | | | | | | | | 0.030 | 0.039 | |
| Seesorb ® 709 | | | | | | | | | | | 1.667 |
| Seesorb ® 703 | | | | | | | | | | | 0.201 |
| Total | 100.000 | 100.000 | 100.000 | 100.000 | 100.000 | 100.000 | 100.000 | 100.000 | 100.000 | 100.000 | 100.000 |

The compositions were prepared according to the following steps:
1) Releasing agent (Zelec® UN), catalyst (DMC), UV absorbers (Seesorb® 701) and blue cut molecule of Formula (I) were dissolved in ISO2 monomer in a Duran® bottle at room temperature. After becoming homogeneous, the mixture was degassed under vacuum
2) The resulting mixture was pre-cooled at 2° C. and placed in a N₂ atmosphere.
3) THIO2 monomer was added and the mixture was stirred at 2° C. until a homogeneous mixture was formed, followed by degassing and purging with N₂ before monomer filling steps.

### Example 4: Preparation of Ophthalmic Lenses with Refractive Index 1.67

Plano glass molds were assembled by taping method to accommodate lens with 2 mm center thickness. The monomer mixture of example 3 was then injected into the prepared assembly using clean syringe and thermally polymerized using specific temperature profile as shown in Table 3.

TABLE 3
Temperature profile for BCT lenses with refractive index 1.67

| SEGMENT | 1 | 2 | 3 | 4 | 5 | 6 | 7 | 8 | 9 | 10 |
|---|---|---|---|---|---|---|---|---|---|---|
| Duration (h) | 0.5 | 1.0 | 1.0 | 1.0 | 1.0 | 1.0 | 1.0 | 1.0 | 1.0 | 1.0 |
| T° Begin (° C.) | 20.0 | 20.0 | 20.0 | 20.1 | 20.3 | 20.5 | 21.0 | 21.7 | 22.9 | 24.8 |
| T° End (° C.) | 20.0 | 20.0 | 20.1 | 20.3 | 20.5 | 21.0 | 21.7 | 22.9 | 24.8 | 28.1 |
| | | | | | | | | | | 20/ |
| SEGMENT | 11 | 12 | 13 | 14 | 15 | 16 | 17 | 18 | 19 | Hold |
| Duration (h) | 1.0 | 1.0 | 1.0 | 1.0 | 1.0 | 1.0 | 1.0 | 1.0 | 1.0 | 7.0 |
| T° Begin (° C.) | 28.1 | 33.4 | 42.2 | 56.7 | 80.6 | 120.0 | 120.0 | 120.0 | 90.0 | 75.0 |
| T° End (° C.) | 33.4 | 42.2 | 56.7 | 80.6 | 120.0 | 120.0 | 120.0 | 90.0 | 75.0 | 75.0 |

Lenses were generally disassembled out of the molds and subject to traditional cleaning and annealing process.

### Example 5: Preparation of Compositions to Obtain Ophthalmic Lenses with Refractive Index 1.6

Compositions 9 to 14 according to the invention and comparative compositions Comp. 4 to 6, comprising the following ingredients were prepared. The values expressed in the table below are weight percentages based on the total weight of the composition. The values expressed in the Table 4 below are weight percentages based on the total weight of the composition.

Preparation process of these compositions was as for the compositions 1 to 8 in example 3, in that all additives were dissolved in ISO1 Monomer and then degassed and pre-cooled before adding THIO1 and THIO2monomers.

TABLE 4
Compositions for ophthalmic lenses with refractive index 1.6.

| | Inventive examples | | | | | | Comparative examples | | |
|---|---|---|---|---|---|---|---|---|---|
| Composition (%) | 9 | 10 | 11 | 12 | 13 | 14 | Comp. 4 | Comp. 5 | Comp. 6 |
| ISO1 | 49.93 | 49.93 | 49.92 | 49.92 | 49.93 | 49.93 | 49.85 | 49.83 | 49.02 |
| THIO1 | 23.59 | 23.59 | 23.58 | 23.58 | 23.59 | 23.59 | 23.55 | 23.54 | 23.15 |
| THIO2 | 25.16 | 25.16 | 25.16 | 25.16 | 25.16 | 25.16 | 25.12 | 25.11 | 24.70 |
| Stan DMC | 0.04 | 0.04 | 0.04 | 0.04 | 0.04 | 0.04 | 0.04 | 0.04 | 0.04 |
| Zelec ® UN | 0.07 | 0.07 | 0.07 | 0.07 | 0.07 | 0.07 | 0.07 | 0.07 | 0.07 |
| Seesorb ® 709 | 1.18 | 1.18 | 1.18 | 1.18 | 1.18 | 1.18 | 1.18 | 1.18 | 1.94 |
| Seesorb ® 703 | 0.01 | 0.01 | 0.01 | 0.01 | 0.01 | 0.01 | 0.01 | 0.01 | 0.12 |
| I-1 | 0.01 | | | | | | | | |
| I-2 | | 0.01 | | | | | | | |
| I-3 | | | 0.030 | 0.035 | | | | | |
| I-4 | | | | | 0.010 | | | | |
| I-5 | | | | | | 0.010 | | | |
| H-Oxa | | | | | | | 0.172 | 0.222 | |
| Tinuvin | | | | | | | | | 0.969 |
| Total | 100.00 | 100.00 | 100.00 | 100.00 | 100.00 | 100.00 | 100.00 | 100.00 | 100.00 |

### Example 6: Preparation of Ophthalmic Lenses with Refractive Index 1.6

Ophthalmic lenses having a refractive index of 1.6 were prepared using the same procedure as the lenses having a refractive index of 1.67, according to example 4. The specific temperature profile is as shown in Table 5

TABLE 5
Temperature profile for BCT lenses with refractive index 1.6

| SEGMENT | 1 | 2 | 3 | 4 | 5 | 6 | 7 | 8 | 9 | 10 | 11 | 12 |
|---|---|---|---|---|---|---|---|---|---|---|---|---|
| Duration (h) | 1.0 | 1.0 | 1.0 | 1.0 | 1.0 | 1.0 | 1.0 | 1.0 | 1.0 | 1.0 | 1.0 | 1.0 |
| T° Begin (° C.) | 15.0 | 15.1 | 15.2 | 15.4 | 15.7 | 16.2 | 17.0 | 18.4 | 20.6 | 22.0 | 27.0 | 34.0 |
| T° End (° C.) | 15.1 | 15.2 | 15.4 | 15.7 | 16.2 | 17.0 | 18.4 | 20.6 | 22.0 | 27.0 | 34.0 | 45.0 |

| SEGMENT | 13 | 14 | 15 | 16 | 17 | 18 | 19 | 20 | 21 | 22 | 23 |
|---|---|---|---|---|---|---|---|---|---|---|---|
| Duration (h) | 1.0 | 1.0 | 1.0 | 1.0 | 1.0 | 1.0 | 1.0 | 1.0 | 1.0 | 1.0 | 500.0 |
| T° Begin (° C.) | 45.0 | 60.0 | 80.0 | 105.0 | 130.0 | 130.0 | 130.0 | 130.0 | 120.0 | 120.0 | 120.0 |
| T° End (° C.) | 60.0 | 80.0 | 105.0 | 130.0 | 130.0 | 130.0 | 130.0 | 120.0 | 120.0 | 120.0 | 120.0 |

Lenses were generally disassembled out of the molds and subject to traditional cleaning and annealing process.

### Example 7: Optical Properties of the Lenses

The BCT lenses of examples 4 and 6 were optically characterized at lens initial stage and after photo-aging test.

The optical characteristics, at the initial state, of the lenses having a refractive index of 1.67 are shown in Table 6 below:

TABLE 6
Comparison of optical properties of BCT lenses having a refractive index of 1.67, at the initial

| | | Initial stage | | | | | | | | | |
|---|---|---|---|---|---|---|---|---|---|---|---|
| | | Content | | | | | | | | | |
| | Lenses | (% wt) | UV_{cut} | TvD65 | YI | L* | a* | b* | C* | h* | BVC B′ (%) |
| Inventive | 1 | 0.004 | 409 | 87.8 | 11.5 | 95.0 | −4.6 | 9.6 | 10.6 | 115.9 | 53.5 |
| | 2 | 0.002 | 404 | 87.7 | 9.8 | 94.9 | −4.0 | 8.2 | 9.1 | 115.9 | 48.5 |
| | 3 | 0.008 | 408 | 88.1 | 6.0 | 95.1 | −2.5 | 5.2 | 5.8 | 115.9 | 36.0 |
| | 4 | 0.012 | 410 | 88.1 | 7.0 | 95.1 | −3.0 | 6.1 | 6.8 | 116.1 | 39.9 |
| | 5 | 0.006 | 411 | 87.8 | 9.5 | 95.0 | −4.0 | 8.1 | 9.0 | 116.0 | 48.7 |
| | 6 | 0.003 | 408 | 87.8 | 12.0 | 95.0 | −4.8 | 9.9 | 11.0 | 115.9 | 54.7 |
| | 7 | 0.01 | 412 | 88.0 | 10.2 | 95.1 | −4.2 | 8.6 | 9.6 | 116.0 | 50.3 |
| | 8 | 0.01 | 411 | 87.9 | 10.5 | 95.0 | −4.2 | 8.8 | 9.7 | 115.4 | 50.0 |
| Comparative | Comp 1 | 0.030 | 408 | 87.8 | 5.7 | 95.0 | −2.4 | 4.9 | 5.5 | 115.6 | 34.9 |
| | Comp 2 | 0.040 | 410 | 88.0 | 6.2 | 95.1 | −2.7 | 5.4 | 6.1 | 116.0 | 37.1 |
| | Comp 3 | 0.201 | 407 | 88.1 | 6.8 | 95.1 | −2.9 | 5.8 | 6.5 | 116.0 | 38.6 |

The results of the photo-aging tests of the lenses having a refractive index of 1.67 are shown in Table 7 below:

TABLE 7
Comparison of the evolution upon photo-aging test
of BCT lenses having a refractive index of 1.67.

Evolution
(Q-sun, 40 h)

| | | ΔUV_{cut} | |
|---|---|---|---|
| | Lenses | (%) | ΔE |
| Inventive | 1 | −0.5 | 2.4 |
| | 2 | 0.0 | 0.4 |
| | 3 | 0.0 | 0.2 |
| | 4 | 0.0 | 0.1 |
| | 5 | 0.0 | 0.2 |
| | 6 | 0.0 | 0.2 |
| | 7 | 0.0 | 0.1 |
| | 8 | −1.0 | 1.5 |
| Comparative | Comp. 1 | −1.0 | 1.1 |
| | Comp. 2 | −0.7 | 0.8 |
| | Comp. 3 | 0.0 | 0.1 |

Lenses according to the invention, having a refractive index of 1.67 outperform comparative lenses in two senses:
1) lower molecule consumption at the same optical cut because of high absorption extinction coefficient, and
2) superior light resistance on both counts, i.e., optical cut and color retention.

Meanwhile, of the fresh lenses, it maintains great aesthetic comparable to the reference lenses. Specifically, example 3 doped with 1-3 required absorber content about 3.7 times less than that of the comparative Comp. 1 and even less compared to Comp. 3 to accommodate same optical cut performance (408 nm) with similar range of residue color YI (5-6)—no aesthetic trade-off. Besides, property durability of example 3 over photo-aging, defined by degree of optical cut (% ΔUV_{cut} ↓) and color change (ΔE ↓) are greater than those of Comp. 1.

The optical characteristics, at the initial state, of the lenses having a refractive index of 1.6 are shown in Table 8 below:

TABLE 8
Comparison of optical properties of BCT lenses, having a refractive index of 1.6, at the initial

| | | Initial stage | | | | | | | | | |
|---|---|---|---|---|---|---|---|---|---|---|---|
| | | Content | | | | | | | | | |
| | Lenses | (% wt) | UV_{cut} | TvD65 | YI | L* | a* | b* | C* | h* | BVC B′ (%) |
| Inventive | 9 | 0.010 | 416 | 89.7 | 12.0 | 95.8 | −5.1 | 10.2 | 11.4 | 116.5 | 55.6 |
| | 10 | 0.010 | 416 | 89.5 | 12.5 | 95.7 | −5.2 | 10.5 | 11.7 | 116.5 | 56.8 |
| | 11 | 0.030 | 410 | 89.9 | 6.0 | 95.9 | −2.8 | 5.5 | 6.2 | 117.1 | 36.5 |
| | 12 | 0.033 | 411 | 89.9 | 6.2 | 95.9 | −2.9 | 5.7 | 6.4 | 117.2 | 37.7 |
| | 13 | 0.010 | 411 | 89.8 | 7.4 | 95.8 | −3.4 | 6.7 | 7.5 | 117.1 | 42.1 |
| | 14 | 0.010 | 417 | 89.8 | 13.0 | 95.8 | −5.5 | 11.0 | 12.2 | 116.5 | 58.3 |
| Comparative | Comp. 4 | 0.175 | 411 | 89.9 | 6.0 | 95.9 | −2.8 | 5.5 | 6.1 | 117.0 | 36.1 |
| | Comp. 5 | 0.225 | 412 | 89.7 | 6.7 | 95.8 | −3.1 | 6.1 | 6.8 | 116.9 | 38.9 |
| | Comp. 6 | 0.120 | 407 | 89.8 | 5.3 | 95.9 | −2.4 | 4.7 | 5.3 | 116.8 | 33.0 |

The results of the photo-aging tests of the lenses having a refractive index of 1.6 are shown in Table 9 below:

TABLE 9
Comparison of the evolution upon photo-aging test
of BCT lenses having a refractive index of 1.6.

| | | Evolution (Q-sun, 40h) | |
|---|---|---|---|
| | Lenses | ΔUV_{cut} (%) | ΔE |
| Inventive | 9 | −0.2 | 0.7 |
| | 10 | 0.0 | 0.2 |
| | 11 | 0.0 | 0.2 |
| | 12 | 0.0 | 0.1 |
| | 13 | 0.0 | 0.1 |
| | 14 | 0.0 | 0.1 |
| Comparative | Comp. 4 | −0.5 | 1.2 |
| | Comp. 5 | −0.2 | 1.5 |
| | Comp. 6 | 0.0 | 0.0 |

In consistence with lenses having a refractive index of 1.67, lenses according to the invention having a refractive index of 1.6 are more industrial economical, i.e., lower absorber content needed at the same cut range, along with possessing higher light resistance relative to some comparative lens examples.

### Example 8: Optical Properties of the Lenses

In this example, the long-term stability of the lenses was studied.

In addition, the thermal and photo stability of lenses was studied, after long term storage.

### Example 8a: Preparation of the Lenses for Storage Studies

Lenses with refractive index 1.67 were prepared according to the procedure as described in example 3.

The lenses prepared in this example included the following compounds:

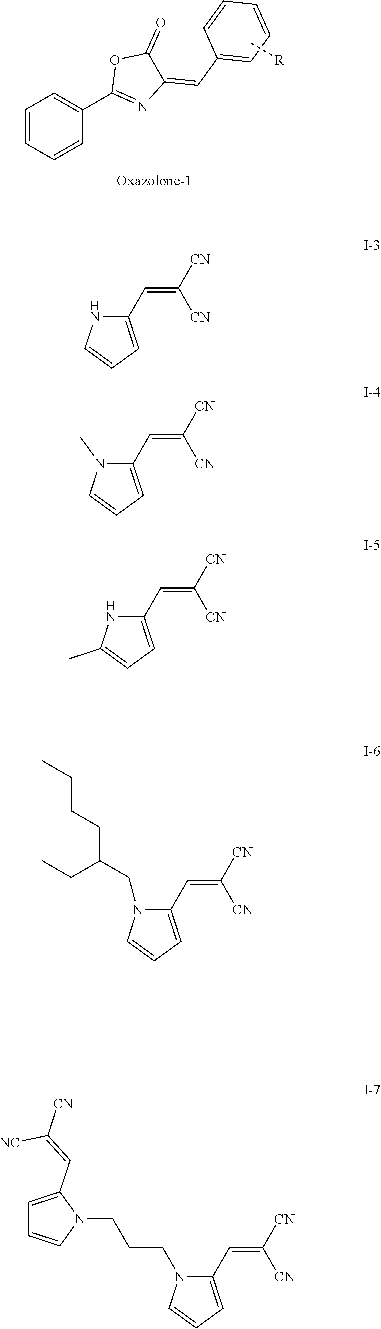

The composition of the lenses is as shown in Table 10.

TABLE 10
Compositions for ophthalmic lenses with refractive index 1.67.

| Composition (%) | 15 | 16 | 17 | 18 | 19 | Comp-7 |
|---|---|---|---|---|---|---|
| ISO2 | 51.388 | 51.388 | 51.387 | 51.386 | 51.390 | 51.351 |
| THIO2 | 47.284 | 47.283 | 47.283 | 47.282 | 47.285 | 47.249 |
| Stan DMC(ppm) | 0.010 | 0.010 | 0.010 | 0.010 | 0.010 | 0.010 |
| Zelec ® (ppm) | 0.079 | 0.079 | 0.079 | 0.079 | 0.079 | 0.079 |
| Seesorb ®701P-UV absorber | 1.233 | 1.233 | 1.233 | 1.233 | 1.233 | 1.232 |
| I-3 | | | | 0.010 | | |
| I-4 | 0.006 | | | | | |
| I-5 | | | | | 0.003 | |
| I-6 | | 0.007 | | | | |
| I-7 | | | 0.008 | | | |
| H-Oxazolone-1 | | | | | | 0.079 |
| Total | | 100.000 | 100.000 | 100.000 | 100.000 | 100.000 |

### Example 8b: Initial Lens Properties Before Storage

The optical characteristics of the lenses as described in example 8a, Table 10, at the initial state, were determined using the procedure as described above, in the same way as for example 3. The results are shown in Table 11 below.

TABLE 11
Initial optical properties of BCT lenses having a refractive index of 1.67

| | Content | Initial optical properties | | | | | | | | |
|---|---|---|---|---|---|---|---|---|---|---|
| Lenses | (% wt) | UVcut | TvD65 | YI | L* | a* | b* | C* | h* | BVC B′ (%) |
| 15 | 0.006 | 411 | 87.8 | 9.4 | 95.0 | −3.9 | 8.0 | 8.9 | 116.0 | 48.2 |
| 16 | 0.007 | 411 | 87.9 | 9.5 | 95.0 | −4.0 | 8.1 | 9.0 | 116.1 | 48.3 |
| 17 | 0.008 | 412 | 87.8 | 11.0 | 95.0 | −4.4 | 9.2 | 10.2 | 115.6 | 51.8 |
| 18 | 0.010 | 409 | 88.1 | 6.4 | 95.1 | −2.7 | 5.6 | 6.2 | 115.9 | 37.4 |
| 19 | 0.003 | 409 | 88.0 | 11.9 | 95.0 | −4.8 | 9.9 | 11.0 | 116.1 | 54.6 |
| Comp-7 | 0.080 | 413 | 88.0 | 7.7 | 95.1 | −3.3 | 6.8 | 7.5 | 116.3 | 42.9 |

### Example 8c: Lens Properties after Storage—General Remarks and Methods

The lenses of example 8a, after casting and post-curing were stored in the dark at ambient temperature, by putting in the envelope for about 1.2 years, and the optical properties were re-measured using UV-vis spectrometer.

The stability of lens properties after storage was examined in terms of color change using ΔE_{lab} parameter, and alteration of blue cut ability using ΔBVCB′ and ΔUV_{cut}.

When ΔE_{Lab}<1%, it is herein defined as acceptable/insignificant change.

When ΔBVCB′<5%, it is defined as acceptable/insignificant change.

When ΔUV_{cut}<0.5%, it is herein defined as acceptable/insignificant change.

For the experiments described herein, ΔE_{lab}, ΔBVCB′ and ΔUV_{cut} were calculated as follows.

$$General calculation for the amplitude of color change ( equation a )$$

$$Δ {E}_{L a b} = \sqrt{{( Δ {L}^{*} )}^{2} + {( Δ {a}^{*} )}^{2} + {( Δ {b}^{*} )}^{2}} \qquad ( Equation a )$$

$$Wherein :$$

$$Δ {L}^{*} = {L}_{( i )}^{*} - {L}_{( {i}_{0} )}^{*}$$

$$Δ {a}^{*} = {a}_{( i )}^{*} - {a}_{( {i}_{0} )}^{*}$$

$$Δ {b}^{*} = {b}_{( i )}^{*} - {b}_{( {i}_{0} )}^{*}$$

i₀ denotes the value at the initial stage and i denotes the value after 1.2 years storage.

### General Calculation for Amplitude of Cut Performance Change (Equation b)

$$Δ {BVCB}^{′} ( \% ) = \frac{{{BVCB}^{′}}_{( i )} - {{BVCB}^{′}}_{( i 0 )}}{{{BVCB}^{′}}_{( i 0 )}} \times 100 \qquad ( Equation b )$$

i₀ denotes the value at the initial stage and i denotes the value after 1.2 years storage.

### General Calculation for Amplitude of Cut Performance Change (Equation c)

$$Δ {UV}_{cut} ( \% ) = \frac{{UV}_{cut ( {i}_{0} )} - {UV}_{cut ( {i}_{0} )}}{{UV}_{cut ( {i}_{0} )}} \times 100 \qquad ( Equation c )$$

i₀ denotes the value at the initial stage and i denotes the value after 1.2 years storage.

### Thermal Stability Tests of Stored Lenses

The above 1.2 years stored lenses were analyzed using UV-vis spectrometer and then subjected to heat at 95° C. for 3 h, mimicking the thermal condition of hard coating process. Subsequently, the lenses were cooled down and left at ambient for at least 1 hour for the relaxation. Then, optical properties of the tested lenses were re-measured using the UV-vis spectrometer.

Shift of color and cut performance of the 1.2 years old lenses upon thermal test were represented by the previous described parameters (ΔE_{lab}, ΔBVCB′ and ΔUV_{cut}), using equations a, b and c.

The general equations a, b and c were adapted to calculate those parameters.

i₀ refers to the value of the 1.2 years old lenses (measuring just before thermal test), and i refer to that of after thermal test.

### Photostability Test of Stored Lenses

The properties of the stored lenses under photo-aging were examined to foresee the robustness of aged lenses over wearing.

The photo-aging test was performed using Q-sun test as described above (for 80 hours).

Likewise, the optical properties of such 1.2 year stocked lenses were measured before and after 80 h cycle of Q-sun test to determine the degree of color and cut performance change over the test. The degree change were quantified following to the equation a, b and c, respectively, where i₀ refer to the value of 1.2 year old lenses, (measuring just before Q-sun test) and i refer to that of lenses after 80 h cycle of Q-sun test.

### Example 8d: Results of the Storage Stability Tests

The results of the above thermal and photo tests are shown in Table 12.

TABLE 12
results of the storage/stability experiments

| | | Test | | | | | | | | |
|---|---|---|---|---|---|---|---|---|---|---|
| | | I: Dark storage | | | II: Thermal stability of ~1.2 | | | II: Photo-stability of ~1.2 | | |
| | | (1.2 years) | | | year aged lenses | | | year aged lenses | | |
| | | | ΔBVCB′ | ΔUVcut | | ΔBVCB′ | ΔUVcut | | ΔBVCB′ | ΔUVcut |
| | Absorber | ΔE < 1 | (%) <5% | (%) <0.5% | ΔE < 1 | (%) <5% | (%) <0.5% | ΔE < 1 | (%) <5% | (%) <0.5% |
| 15 | I-4 | 0.5 | 3.4 | 0.00 | 0.4 | 1.0 | 0.0 | 0.3 | −2.0 | −0.2 |
| 16 | I-6 | 0.3 | 2.5 | 0.00 | 0.2 | 0.5 | 0.0 | 0.3 | −0.9 | −0.2 |
| 17 | I-7 | 0.3 | 0.4 | 0.00 | 0.3 | 0.6 | 0.0 | 2.0 | −1.0 | −1.0 |
| 18 | I-3 | NA | NA | NA | 1.5 | 4.9 | 0.0 | 0.6 | −3.2 | −0.1 |
| 19 | I-5 | NA | NA | NA | NA | NA | NA | NA | NA | NA |
| Comp-7 | Oxazolone | 0.3 | 2.5 | −0.20 | 1.5 | −13.5 | −0.5 | 3.0 | −33.8 | −2.3 |

It follows from Table 12 that lenses 15-18 according to the invention overall showed improved thermal and photo stability after storage (tests II and III), as compared to comparative lens comprising oxazolone.

In particular, lenses made of compounds I-4, I-6 and I-7 showed a particularly high stability.

It was shown that these lenses, comprising N-alkyl-substituted pyrrole acrylonitrile, are particularly stable upon storage and post-thermal and photostability conditions, when compared to comparative lenses comprising oxazolone and lenses comprising a pyrrole acrylonitrile which is non-substituted on nitrogen (I-3 and I-5).

Specifically, lenses cast with N-alkyl substituted I-4 and I-6 displayed high robustness on all counts; acceptable color and cut ability over time and post-conditioning, i.e., thermal- and photo-step after aging time.

1. An ophthalmic lens comprising:
a plastic base, and
a compound of Formula (I) or a salt thereof, in particular a hydrochloride salt or a sulfate salt 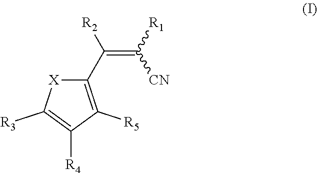
wherein:
R₁ is chosen from —CN, —CONH₂, —CO₂R₆ or —SO₂—(C₁-C₁₂)alkyl, R₂ is, independently from R₃, R₄ and R₅, chosen from H, F, Cl, Br, I, —OH, —OR₇, —SH, —SR8, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl, R₃, R₄ and R₅ are independently chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
X is NH, NR₁₅, O, or S,
R₆, R₇, R₈, R₉, R₁₀, R₁₁ R₁₂, R₁₃ and R₁₄ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl,
R₁₅ is chosen from (C₁-C₁₂)alkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl, (5 to 10 membered)heteroaryl-(C₁-C₁₂)alkyl, said alkyl being optionally substituted by one to three R₁₆ groups,
R₁₆ is chosen from halogen atoms, (C₁-C₁₂)alkoxy, (C₆-C₁₈)aryl,
or wherein R₁₅ is a structure of Formula a: 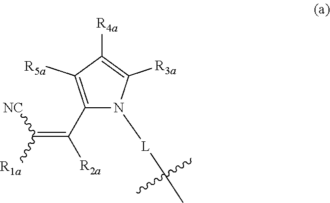
in which
L is (C₁-C₁₂)alkylene,
R₁ₐ is chosen from —CN, —CONH₂, —CO₂R₆ₐ or —SO₂—(C₁-C₁₂)alkyl,
R₂ₐ is, independently from R₃ₐ, R₄ₐ and R₅ₐ, chosen from H, F, Cl, Br, I, —OH, —OR₇ₐ, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₃ₐ, R₄ₐ and R₅ₐ are independently chosen from H, F, Cl, Br, I, —OH, —OR₇, —SH, —SR₅ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₆ₐ, R₇ₐ, R₅ₐ, R₉ₐ, R₁₀ₐ, R₁₁ₐ, R₁₂ₐ, R₁₃ₐ and R₁₄ₐ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl,
wherein the aryl or heteroaryl groups are optionally substituted by one or more substituents, in particular by 1 or 2 substituents, said substituents being in particular chosen from:
halogen, in particular F or Cl,
carboxylic ester, in particular methyl ester or ethyl ester,
(C₁-C₁₂)alkyl, in particular methyl, ethyl or iso-propyl,
(C₁-C₁₂)alkoxy, in particular methoxy or ethoxy,
carboxylic acid,
nitrile,
amine, in particular —NH₂, NHCH₃, or N(CH₃)₂.

2. The ophthalmic lens according to claim 1, wherein R₁ and R₁ₐ are independently chosen from —CN or —CO₂Et, and/or
wherein R₂, R₂ₐ, R₃, R₃ₐ, R₄, R₄ₐ, R₅ and R₅ₐ are independently chosen from H or (C₁-C₁₂)alkyl, preferably H or CH₃.

3. The ophthalmic lens according to claim 1,
wherein X is NH or NR₁₅, in particular NH or —NCH₃, —NCH₃CH₃—N—CH₂C(CH₂CH₃)(CH₂)₃CH₃, —NC(CH₃)₃, —NCH₂—CF₃, N(CH₂)₂-phenyl, N—(CH₂)₄—F or N—(CH₂)₄—OCH₃,
R₁₅ being as defined in claim 1.

4. The ophthalmic lens according to claim 1,
wherein the compound of Formula (I) is such that:
R₁ is —CN,
and
X is —NR₁₅,
R₁₅ being as defined in claim 1, in particular chosen from —(C₁-C₁₂)alkyl, optionally substituted by one to three R₁₆ groups, in particular chosen from halogen atoms, (C₁-C₁₂)alkoxy, (C₆-C₁₅)aryl,
or a compound of Formula a as defined in claim 1 in which R₁ₐ is —CN, and R₂ₐ, R₃ₐ, R₄ₐ and R₅ₐ are H.

5. The ophthalmic lens according to claim 1,
wherein the compound of Formula (I) is such that:
R₁ is —CN,
R₂, R₃, R₄ and R₅ are H, and
X is —NR₁₅,
R₁₅ being as defined in claim 1, in particular chosen from —(C₁-C₁₂)alkyl, optionally substituted by one to three R₁₆ groups, in particular chosen from halogen atoms, (C₁-C₁₂)alkoxy, (C₆-C₁₈)aryl,
or a compound of Formula a as defined in claim 1 in which R₁ₐ is —CN, and R₂ₐ, R₃ₐ, R₄ₐ and R₅ₐ are H.

6. The ophthalmic lens according to claim 1, wherein the compound of Formula (I) is chosen from compounds of Formulae I-1 to I-7:

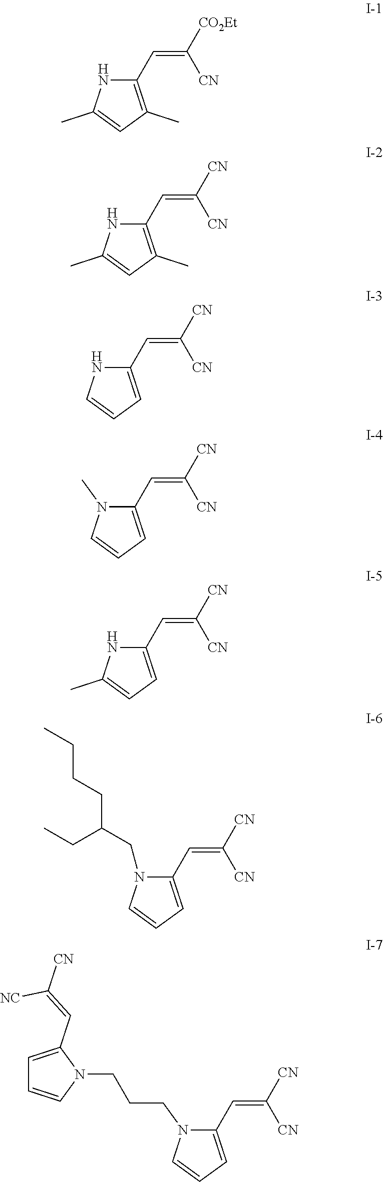

7. The ophthalmic lens according to claim 1, wherein the compound of Formula (I) has a maximum absorption wavelength (λₘₐₓ) lower than or equal to 400 nm, as measured on a 20 ppm solution of compound of Formula I in an organic solvent, in particular in tetrahydrofuran, said maximum absorption wavelength being in particular in the range of from 350 to 400 nm, more in particular of from 370 to 400 nm.

8. The ophthalmic lens according to claim 7, wherein at the maximum absorption wavelength (λₘₐₓ), the compound of Formula (I) has a molar absorption extinction coefficient (c) higher than 30,000 M⁻¹cm⁻¹, said molar absorption extinction coefficient (c) being in particular in the range of from 30,000 to 80,000 M⁻¹cm⁻¹, more in particular in the range of from 30,000 to 70,000 M⁻¹cm⁻¹, even more in particular in the range of from 30,000 to 45,000 M⁻¹cm⁻¹.

9. The ophthalmic lens according to claim 1, wherein the compound of Formula (I) is included in the plastic base or is included in a separate layer coated on a surface of the plastic base.

10. The ophthalmic lens according to claim 1, wherein the amount of compound of Formula (I) is in the range of from 0.001 to 2%, in particular of from 0.001 to 1%, more in particular of from 0.001 to 0.1%, even more in particular of from 0.01 to 0.05% by weight based on the weight of the plastic base when the compound of Formula (I) is included in the plastic base or on the weight of a separate layer when the compound of Formula (I) is included in a separate layer coated on a surface of the plastic base.

11. The ophthalmic lens according to claim 1, wherein the ophthalmic lens further comprises a benzotriazole UV-absorber or a mixture thereof, preferably a benzotriazole UV-absorber selected from 2-(2-hydroxy-5-tert-octylphenyl)-2H-benzotriazole, 2-(3-tert-butyl-2-hydroxy-5-methylphenyl)-5-chloro-2H-benzotriazole, and mixtures thereof, in particular wherein the total amount of benzotriazole UV-absorber is in the range of from 0.001 to 2%, in particular of from 0.01 to 1.5%, more particularly of from 0.1 to 1.25%, by weight based on the weight of the plastic base.

12. The ophthalmic lens according to claim 1, wherein the plastic base is a thermoset resin or a thermoplastic resin, in particular a thermoset resin, in particular comprising a copolymer of at least one polythiol and a polyisocyanate, and/or wherein the plastic base has a refractive index of from 1.4 to 1.9, in particular of from 1.49 to 1.74.

13. The ophthalmic lens according to claim 1, wherein UV_{cut} is higher than 403 nm, in particular comprised in the range of from 403-420 nm, more in particular comprised in the range of from 404-411 nm, and/or
wherein the transmittance TvD65 of the ophthalmic lens is greater than 85%, in particular greater than 87%, and/or
wherein the yellow index YI is lower than 14, and/or
wherein the BVC B′ value is greater than 25%, in particular greater than 30%, more in particular greater than 35%.

14. The ophthalmic lens according to claim 1, wherein the optical cut change ΔUV_{cu}t after exposure of the ophthalmic lens for 40 hours to light in a Q-SUN Xe-3 xenon test chamber is less than 2%, in particular less than 1%, and or wherein the perceived color difference ΔE_{lab} after exposure of the ophthalmic lens for 40 hours to light in a Q-SUN Xe-3 xenon test chamber is less than 2.5, in particular less than 1.

15. A process for preparing the ophthalmic lens according to claim 1, comprising the steps of:
a) providing monomers or oligomers from which the plastic base can be prepared;
b) mixing the monomers or oligomers, the compound of Formula (I) and a catalyst suitable for the polymerization of the monomers and oligomers to form a polymerizable liquid composition;
c) curing the polymerizable liquid composition.

16. (canceled)

17. A composition comprising a plastic base and a compound of formula (IA), 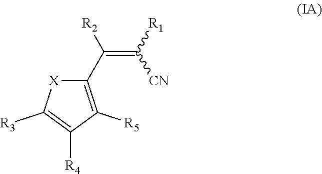
wherein:
R₁ is chosen from —CN, —CONH₂, —CO₂R₆ or —SO₂—(C₁-C₁₂)alkyl,
R₂ is, independently from R₃, R₄ and R₅, chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, or (5 to 10 membered)heteroaryl-(C₁-C₁₂)alkyl,
R₃, R₄ and R₅ are independently chosen from H, F, Cl, Br, I, —OH, —OR₇, —SH, —SR₈, —SOR₉, SO₂R₁₀, —NH₂, —NHR₁₁, —N(R₁₂)₂, —NHCOR₁₃, —NHSO₂OR₁₄, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
X is NH, NR₁₅, O, or S,
R₆, R₇, R₈, R₉, R₁₀, R₁₁, R₁₂, R₁₃ and R₁₄ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl,
R₁₅ is chosen from (C₁-C₁₂)alkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl, (5 to 10 membered)heteroaryl-(C₁-C₁₂)alkyl, said alkyl being optionally substituted by one to three R₁₆ groups,
R₁₆ is chosen from halogen atoms, (C₁-C₁₂)alkoxy, (C₆-C₁₈)aryl,
or wherein R₁₅ is a structure of Formula b: 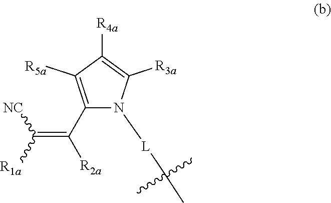
in which
L is (C₁-C₁₂)alkylene,
R₁ₐ is chosen from —CN, —CONH₂, —CO₂R₆ₐ or —SO₂—(C₁-C₁₂)alkyl,
R₂ₐ is, independently from R₃ₐ, R₄ₐ and R₅ₐ, chosen from H, F, Cl, Br, I, —OH, —OR₇ₐ, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₃ₐ, R₄ₐ and R₅ₐ are independently chosen from H, F, Cl, Br, I, —OH, —OR₇, —SH, —SR₈ₐ, —SOR₉ₐ, SO₂R₁₀ₐ, —NH₂, —NHR₁₁ₐ, —N(R₁₂ₐ)₂, —NHCOR₁₃ₐ, —NHSO₂OR₁₄ₐ, —NO₂, —CN, (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)heteroaryl-(C₁-C₁₂)alkyl,
R₆ₐ, R₇ₐ, R₅ₐ, R₉ₐ, R₁₀ₐ, R₁₁ₐ, R₁₂ₐ, R₁₃ₐ and R₁₄ₐ are independently chosen from (C₁-C₁₂)alkyl, (C₁-C₁₂)haloalkyl, (C₂-C₁₂)alkenyl, (C₂-C₁₂)alkynyl, (C₁-C₁₂)heteroalkyl, (C₃-C₁₂)cycloalkyl, (C₃-C₁₂)heterocycloalkyl, (C₆-C₁₈)aryl, (C₆-C₁₈)aryl-(C₁-C₁₂)alkyl, (5 to 10 membered)heteroaryl or (5 to 10 membered)-heteroaryl-(C₁-C₁₂)alkyl,
wherein the aryl or heteroaryl groups are optionally substituted by one or more substituents, in particular by 1 or 2 substituents, said substituents being in particular chosen from:
halogen, in particular F or Cl,
carboxylic ester, in particular methyl ester or ethyl ester,
(C₁-C₁₂)alkyl, in particular methyl, ethyl or iso-propyl,
(C₁-C₁₂)alkoxy, in particular methoxy or ethoxy,
carboxylic acid,
nitrile,
amine, in particular —NH₂, NHCH₃, or N(CH₃)₂,
said plastic base being as defined in claim 1.

18. (canceled)

19. A compound chosen from: 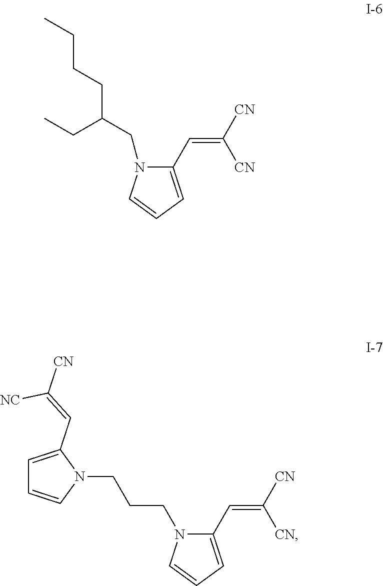 [SMILES: CCCCC(CC)Cn1cccc1C=C(C#N)C#N ; N#CC(C#N)=Cc1cccn1CCCn1cccc1C=C(C#N)C#N]
or salts thereof.
